(19) United States
(12) Patent Application Publication
(10) Pub. No.: US 2018/0147223 A1
(43) Pub. Date: May 31, 2018

(54) BOTULINUM NEUROTOXIN INHIBITORS

(71) Applicant: Prime Bio, Inc, North Dartmouth, MA (US)

(72) Inventors: Bal Ram Singh, Dartmouth, MA (US); Kruti Patel, Dartmouth, MA (US)

(73) Assignee: Prime Bio, Inc, North Dartmouth, MA (US)

(21) Appl. No.: 15/362,959

(22) Filed: Nov. 29, 2016

(51) Int. Cl. A61K 31/704; A61K 31/366; A61K 31/353; A61K 31/11; A61K 31/122; A61K 31/385; A61K 31/4745

(52) U.S. Cl. CPC A61K 31/704; A61K 31/366; A61K 31/353; A61K 31/4745; A61K 31/122; A61K 31/385; A61K 31/11

(57) ABSTRACT

Present invention discloses a method of treating an individual suffering from botulism by inhibiting botulinum neurotoxins.

### FIELD OF INVENTION

Present invention discloses a method of treating an individual suffering from botulism using natural or synthetic compounds.

### BACKGROUND OF INVENTION

The present invention relates to discovery and development of inhibitors for botulinum neurotoxin. Botulinum Neurotoxins (BoNTs) are proteins responsible for the deadly paralytic disease called botulism. Extreme toxicity, ease of production and lack of antidotes against BoNT makes it a category A, biothreat agent according to the United States Center of Disease Control and prevention. These deadly toxins, in minute quantities, estimated human i.v. LD50 of 0.1 ng/kg body weight, cause fatal flaccid paralysis by blocking neurotransmitter release.

BoNT is designated as a “Category A” agent on the National Institute of Allergy and Infectious Diseases (NIAID)-priority-pathogen list and poses a great threat to public health. Due to their high toxicity and relatively easy production, BoNTs create maximum fear among populations concerned with bioterror agents. Contamination of restaurant, catered or commercial food stuffs, or beverages could cause illness in a large number of consumers. Aerosol exposure of BoNTs does not occur naturally, but could be attempted by bioterrorists to achieve widespread effect. A single gram of crystalline toxin, evenly dispersed and inhaled, would kill more than one million people, although technical factors would make such dissemination difficult. A more realistic scenario suggests that less than one gram of BoNT, if distributed into a food supply, such as milk, could cause more than 100,000 casualties. Currently, there is no effective antidote available, except the equine antitoxin sera, and no safe prophylaxis against botulism. There is an urgent need to develop both prophylactic and therapeutic agents against BoNT. The challenge of developing a more effective treatment for botulism has been recognized by NIAID, and has been among NIAID's highest priority.

BoNTs are produced by the bacteria C. botulinum and are released into the medium after bacterial lysis, as an inactive 150 kDa single polypeptide chain. Therefore seven serotypes of the botulinum neurotoxins. These are botulinum neurotoxin A, B, C, D, E F and G. The botulinum neurotoxin is 150 kDa, and the toxin produced in the bacteria is in the form of a complex, containing the neurotoxin and neurotoxin associated proteins (NAPs). The 150 kDa protein is post-translationally proteolyzed (nicked) by bacterial proteases to form the biologically active di-chain neurotoxin, composed of a 100 kDa heavy chain (HC) and a 50 kDa light chain (LC), linked through a disulfide bond (FIG. 1) and non-covalent protein interactions. All seven serotypes have similar mechanism of action facilitate by three common protein domains with specific functions, which work together to establish toxicity. An active toxin consist of:
i) a neuro-specific receptor binding domain—50 kDa carboxy terminal heavy chain (HCC),
ii) a membrane translocation domain—50 kDa amino-terminal heavy chain (HCN),
iii) a catalytic domain—50 kDa zinc endo-peptidase light chain (LC).

Light chain and amino terminal heavy chain are bridged by a single disulfide bond. Naturally botulism can cause three ways:
(i) food borne botulism caused by ingestion of toxin from foods
(ii) (ii) through wound caused by contamination of a wound by BoNT producing spores/bacteria; and
(iii) infant botulism caused by colonization of the digestive tract by the bacterium in children.

Among seven serotypes of botulinum neurotoxins types A, B, E and in rare case F cause botulism in humans. Types C and D cause disease in birds and mammals. Type G, identified in 1970, has not yet been confirmed as a cause of illness in humans or animals.

Among all seven serotypes of botulinum neurotoxins BoNT/A is the most potent and it takes more than six months to recover from botulism caused by BoNT/A. Further, the only available therapy for BoNT is an equine antitoxin antibody or/and a protracted respiratory support system. Even the antibody treatment can only prevent further exposure of the toxin and cannot treat the already intoxicated neurons.

The long lasting endopeptidase activity of the BoNTs is a critical biological activity inside the nerve cell, as it catalyzes proteolysis of the SNARE proteins involved in the exocytosis of acetylcholine, thus causing muscle paralysis. Therefore, there is an urgent need to identify candidates which can inhibitor BoNT's endopeptidase activity, which could be developed into an ultimate therapeutics for treating botulism.

### OBJECT OF THE INVENTION

The main object of the present invention to discloses a method of treating an individual suffering from botulism using natural or synthetic compounds.

### SUMMARY OF INVENTION

The present invention will now be further described. In the following passages different aspects of the invention are defined in more detail. Each aspect defined below may be combined with any other aspect or aspects unless clearly indicated to the contrary. In particular, any feature indicated as being preferred or advantageous may be combined with any other feature or features indicated as being preferred or advantageous.

The main aspects of present invention is a method of treating an individual suffering from botulism comprising:

Administering to the subject a composition comprising a therapeutically effective amount of compound 1-10 or its pharmaceutically acceptable salts or derivatives either alone or combination;
wherein the compound 1-10 are;

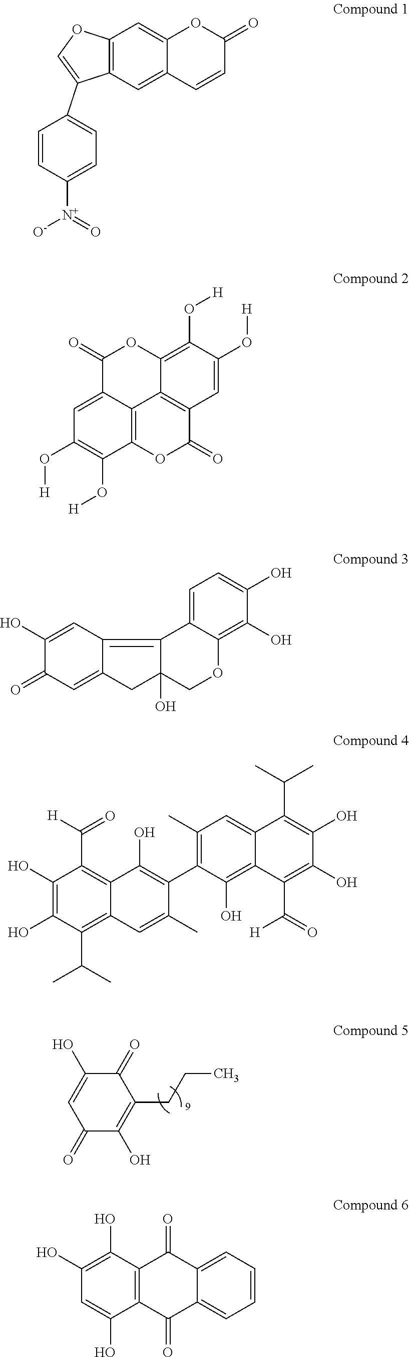

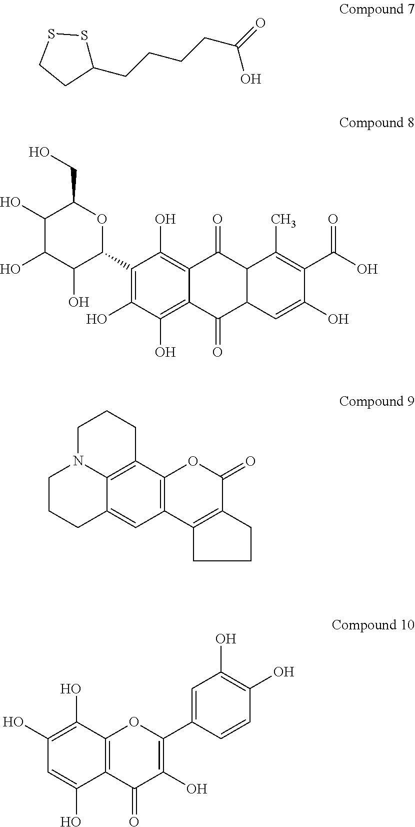 [SMILES: O=C(O)CCCCC1CCSS1 ; CC1=C(C(=O)O)C(O)=CC2C(=O)c3c(O)c(O)c([C@H]4O[C@H](CO)C(O)C(O)C4O)c(O)c3C(=O)C12 ; O=c1oc2c3c4c(cc2c2c1CCC2)CCCN4CCC3 ; O=c1c(O)c(-c2ccc(O)c(O)c2)oc2c(O)c(O)cc(O)c12]

In another embodiment the compound 1 is 3-(4-nitrophenyl)-7H-furo[3,2-g][1]benzopyran-7-one known as psoralen nitro benzene (PNB). The pharmaceutically acceptable salt means salts prepared by alkali metal and alkaline earth metal hydroxides or carbonates or bicarbonates or with any organic amines. The derivatives means, in compound one the nitro group may be replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. The lactum may be opened to form a carboxylic acid group or converted to ester amide, aldehyde. Further the rings may be substituted with amine, alkylated amines, acyl groups, substituted or unsubstituted alkyl groups with maximum four carbons, hydroxyl group, halogen, aldehyde, carboxylic acid, ester, amide, substituted amide etc. The carboxylic acid group obtained by opening lactum may be converted to corresponding salts prepared by alkali metal and alkaline earth metal hydroxides or carbonates or bicarbonates or with any organic amines.

In another embodiment the compound 2 is 2,3,7,8-Tetrahydroxy-chromeno[5,4,3-cde]chromene-5,10-dione known as ellagic acid. The pharmaceutically acceptable salt means salts prepared by alkali metal and alkaline earth metal hydroxides or carbonates or bicarbonates or with any organic amines. The derivatives means, in compound 2 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. One or both the lactum may be opened to form a carboxylic acid group or converted to ester amide, aldehyde. Further the rings may be substituted with amine, alkylated amines, acyl groups, substituted or unsubstituted alkyl groups with maximum four carbons, hydroxyl group, halogen, aldehyde, carboxylic acid, ester, amide, substituted amide etc. The carboxylic acid group obtained by opening lactum, which may be converted to corresponding salts prepared by alkali metal and alkaline earth metal hydroxides or carbonates or bicarbonates or with any organic amines.

In another embodiment the compound 3 is 3, 4, 6a, 10-Tetrahydroxy-6,7-dihydroindeno[2,1-c]chromen-9-one known as Hematein. The pharmaceutically acceptable salt means salts prepared by alkali metal and alkaline earth metal hydroxides or carbonates or bicarbonates or with any organic amines. The derivatives means, in compound 3 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

In another embodiment the compound 4 is 2, 2′-Bis(formyl-1,6,7-trihydroxy-5-isopropyl-3-methylnaphthalene) known as Gossypol. The pharmaceutically acceptable salt means salts prepared by alkali metal and alkaline earth metal hydroxides or carbonates or bicarbonates or with any organic amines. The derivatives means, in compound 4 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. Further aldehyde may be converted to carboxylic acid, ester amide, alcohol or its salts.

In another embodiment the compound 5 is 2,5-dihydroxy-3-undecylcyclohexa-2,5-diene-1,4-dione. The pharmaceutically acceptable salt means salts prepared by alkali metal and alkaline earth metal hydroxides or carbonates or bicarbonates or with any organic amines. The derivatives means, in compound 5 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

In another embodiment the compound 6 is 1, 2, 4-trihydroxyanthracene-9,10-dione known as Purpurin. The pharmaceutically acceptable salt means salts prepared by alkali metal and alkaline earth metal hydroxides or carbonates or bicarbonates or with any organic amines. The derivatives means, in compound 6 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

In another embodiment the compound 7 is 5-(1,2-Dithiolan-3-yl)-pentanoic acid known as Lipoic Acid or thioctic aid. The pharmaceutically acceptable salt means salts prepared by alkali metal and alkaline earth metal hydroxides or carbonates or bicarbonates or with any organic amines.

In another embodiment the compound 8 is carminic acid. The pharmaceutically acceptable salt means salts prepared by alkali metal and alkaline earth metal hydroxides or carbonates or bicarbonates or with any organic amines. The derivatives means, in compound 8 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. Further the compound may be with or without sugar group and the sugar group may be replaced by any other sugar moiety such as arabinose, fructose, glucose etc.

In another embodiment the compound 9 is 2,3,6,7,10,11-Hexahydro-1H,5H-cyclopenta[3,4][1]benzopyrano[6,7,8-ij]quinolizin-12(9H)-one. The derivatives means, in compound 9 the ring may be substituted with functional group such as amine, alkylated amines, acyl groups, substituted or unsubstituted alkyl groups with maximum four carbons, hydroxyl group, halogen, aldehyde, carboxylic acid, ester, amide, substituted amide etc. The carboxylic acid group obtained by opening lactum may be converted to corresponding salts prepared by alkali metal and alkaline earth metal hydroxides or carbonates or bicarbonates or with any organic amines.

In another embodiment the compound 10 is 2-(3, 4-dihydroxyphenyl)-3, 5, 7, 8-tetrahydroxychromen-4-one known as gossypetin, which is a flavonoid. The pharmaceutically acceptable salt means salts prepared by alkali metal and alkaline earth metal hydroxides or carbonates or bicarbonates or with any organic amines. The derivatives means, in compound 5 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

In another aspects of present invention disclose that the compound 1-10 optionally with pharmaceutically acceptable diluent, excipient or carrier Further present invention disclose that the composition further comprises pharmaceutically acceptable binder(s), lubricant(s), suspending agent(s), coating agent(s), solubilizing agent(s).

In yet another aspect of present invention disclose that the compound 1-10 further delivered in combination with liposomes or nanoparticle linked to detoxified recombinant BoNT.

In another aspects of present invention disclose that the botulism caused by botulinum neurotoxin produced by clostridium botulinum.

In further aspects of present invention disclose that the compounds 1-10 inhibit the botulinum neurotoxin.

In another aspects of present invention disclose that the botulinum neurotoxin is type A, B, C, D, E, F, G.

In another aspects of present invention disclose that the botulinum neurotoxin is more preferably A, B, E and F.

In yet another aspects of present invention disclose that the diluent is selected from the group consisting of ethanol, glycerol, DMSO water or mixture thereof.

In another aspects of present invention disclose that the carrier is selected from group consisting of suitable carriers include lactose, starch, glucose, methyl cellulose, magnesium stearate, mannitol, sorbitol.

In another aspects of present invention disclose that the binders selected from the group consisting of starch, gelatin, natural sugars such as glucose, anhydrous lactose, free-flow lactose, beta-lactose, corn sweeteners, natural and synthetic gums.

In further aspects of present invention disclose that the natural and synthetic gums selected from the group consisting of acacia, tragacanth or sodium alginate, carboxymethyl cellulose and polyethylene glycol.

In another aspects of present invention disclose that the lubricant is selected from the group consisting of sodium oleate, sodium stearate, magnesium stearate, sodium benzoate, sodium acetate, sodium chloride.

In another aspects of present invention disclose that the therapeutically effective amount is in the range of <1 to 10 μM per 50 nM of Botulinum neurotoxin.

In another aspects of present invention disclose that the compound is administered by oral administration, nasal administration, topical administration, parenteral administration, rectal administration, systemic administration, intramuscular administration, or intravenous administration.

In another aspects of present invention disclose that the compound is administered more preferably by oral administration or nasal administration.

### BRIEF DESCRIPTION OF DRAWINGS

FIG. 1A-1D shows IC₅₀ graph of compounds 1-10, showing % inhibitor of the LCA endopeptidase activity vs. concentration of inhibitor concentration.

FIG. 2 shows inhibition of LCA endopeptidase activity against SNAG substrate by the inhibitor.

FIG. 3 shows inhibition kinetics of MLN 4493 (Psoralen nitro benzene) with the LC/A. The concentrations used are 25 μM, 12.5 μM and 6.25 μM.

FIG. 4 shows effect of nitro derivative of psoralen on M17 cells viability.

FIG. 5 shows cleavage of SNAP-25 by BoNT/A in M17 cells in the absence and presence of different concentrations of the inhibitors.

FIG. 6 shows endogenous SNAP-25 cleavage in M-17-TR2 cells

### DETAILED DESCRIPTION OF INVENTION

The main aspect of present invention is a method of treating an individual suffering from botulism comprising:

administering to the subject a composition comprising a therapeutically effective amount of compound 1-10 or its pharmaceutically acceptable salts or derivatives either alone or combination;
wherein the compound 1-10 are;

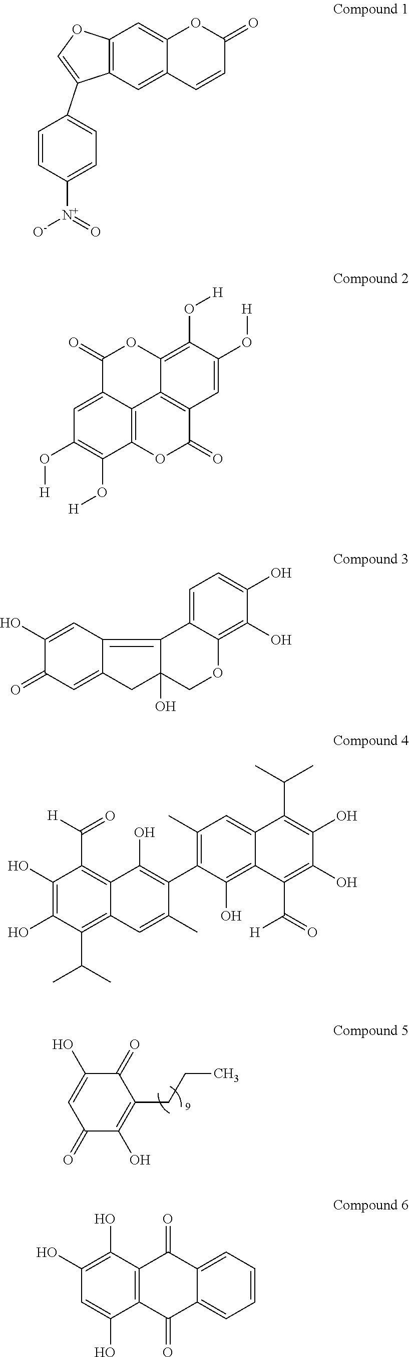

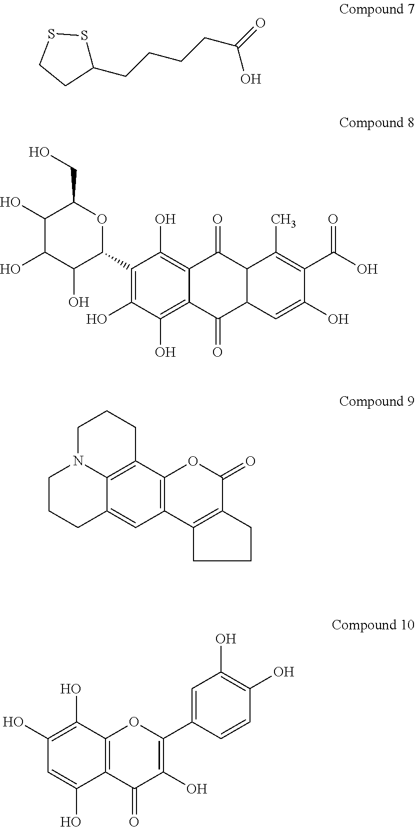 [SMILES: O=C(O)CCCCC1CCSS1 ; CC1=C(C(=O)O)C(O)=CC2C(=O)c3c(O)c(O)c([C@H]4O[C@H](CO)C(O)C(O)C4O)c(O)c3C(=O)C12 ; O=c1oc2c3c4c(cc2c2c1CCC2)CCCN4CCC3 ; O=c1c(O)c(-c2ccc(O)c(O)c2)oc2c(O)c(O)cc(O)c12]

Compound 1 is 3-(4-nitrophenyl)-7H-furo[3,2-g][1]benzopyran-7-one known as psoralen nitro benzene (PNB). The pharmaceutically acceptable salt means salts prepared by alkali metal and alkaline earth metal hydroxides or carbonates or bicarbonates or with any organic amines. The derivatives means, in compound one the nitro group may be replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. The lactum may be opened to form a carboxylic acid group or converted to ester amide, aldehyde. Further the rings may be substituted with amine, alkylated amines, acyl groups, substituted or unsubstituted alkyl groups with maximum four carbons, hydroxyl group, halogen, aldehyde, carboxylic acid, ester, amide, substituted amide etc. The carboxylic acid group obtained by opening lactum may be converted to corresponding salts prepared by alkali metal and alkaline earth metal hydroxides or carbonates or bicarbonates or with any organic amines.

Compound 2 is 2,3,7,8-Tetrahydroxy-chromeno[5,4,3-cde]chromene-5,10-dione known as ellagic acid. The pharmaceutically acceptable salt means salts prepared by alkali metal and alkaline earth metal hydroxides or carbonates or bicarbonates or with any organic amines. The derivatives means, in compound 2 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. One or both the lactum may be opened to form a carboxylic acid group or converted to ester amide, aldehyde. Further the rings may be substituted with amine, alkylated amines, acyl groups, substituted or unsubstituted alkyl groups with maximum four carbons, hydroxyl group, halogen, aldehyde, carboxylic acid, ester, amide, substituted amide etc. The carboxylic acid group obtained by opening lactum, which may be converted to corresponding salts prepared by alkali metal and alkaline earth metal hydroxides or carbonates or bicarbonates or with any organic amines. Compound 3 is 3, 4, 6a, 10-Tetrahydroxy-6,7-dihydroindeno[2,1-c]chromen-9-one known as Hematein. The pharmaceutically acceptable salt means salts prepared by alkali metal and alkaline earth metal hydroxides or carbonates or bicarbonates or with any organic amines. The derivatives means, in compound 3 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

Compound 4 is 2, 2′-Bis(formyl-1,6,7-trihydroxy-5-isopropyl-3-methylnaphthalene) known as Gossypol. The pharmaceutically acceptable salt means salts prepared by alkali metal and alkaline earth metal hydroxides or carbonates or bicarbonates or with any organic amines. The derivatives means, in compound 4 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. Further aldehyde may be converted to carboxylic acid, ester amide, alcohol or its salts.

Compound 5 is 2,5-dihydroxy-3-undecylcyclohexa-2,5-diene-1,4-dione. The pharmaceutically acceptable salt means salts prepared by alkali metal and alkaline earth metal hydroxides or carbonates or bicarbonates or with any organic amines. The derivatives means, in compound 5 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

Compound 6 is 1, 2, 4-trihydroxyanthracene-9,10-dione known as Purpurin. The pharmaceutically acceptable salt means salts prepared by alkali metal and alkaline earth metal hydroxides or carbonates or bicarbonates or with any organic amines. The derivatives means, in compound 6 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

Compound 7 is 5-(1,2-Dithiolan-3-yl)-pentanoic acid known as Lipoic Acid or thioctic aid. The pharmaceutically acceptable salt means salts prepared by alkali metal and alkaline earth metal hydroxides or carbonates or bicarbonates or with any organic amines.

Compound 8 is carminic acid. The pharmaceutically acceptable salt means salts prepared by alkali metal and alkaline earth metal hydroxides or carbonates or bicarbonates or with any organic amines. The derivatives means, in compound 8 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc. Further the compound may be with or without sugar group and the sugar group may be replaced by any other sugar moiety such as arabinose, fructose, glucose etc.

Compound 9 is 2,3,6,7,10,11-Hexahydro-1H,5H-cyclopenta[3,4][1]benzopyrano[6,7,8-ij]quinolizin-12(9H)-one. The derivatives means, in compound 9 the ring may be substituted with functional group such as amine, alkylated amines, acyl groups, substituted or unsubstituted alkyl groups with maximum four carbons, hydroxyl group, halogen, aldehyde, carboxylic acid, ester, amide, substituted amide etc. The carboxylic acid group obtained by opening lactum may be converted to corresponding salts prepared by alkali metal and alkaline earth metal hydroxides or carbonates or bicarbonates or with any organic amines.

Compound 10 is 2-(3, 4-dihydroxyphenyl)-3, 5, 7, 8-tetrahydroxychromen-4-one known as gossypetin, which is a flavonoid. The pharmaceutically acceptable salt means salts prepared by alkali metal and alkaline earth metal hydroxides or carbonates or bicarbonates or with any organic amines. The derivatives means, in compound 5 the at least one or more of hydroxyl group is replaced by any other functional group such as amine, alkylated amines, acyl groups, substituted or unsubstituted alkyl groups with maximum four carbons, hydroxyl group, halogen, aldehyde, carboxylic acid, ester, amide, substituted amide etc.

The inhibitory activity of these compounds tested using a truncated form of a membrane protein SNAP-25 and a 13 amino acid long fluorescence resonance energy transfer (FRET) based peptide was used as the substrate to assay BoNT/A LC endopeptidase activity. β-alanine was added to the C-terminus for the efficient labeling of the fluorophore (Fluorescein-5-isothiocyanate, FITC). The sequence of the peptide is FITC-b (Ala)-Thr-(D-Arg)-Ile-Asp-Gln-Ala-Asn-Gln-Arg-Ala-Thr-Lys (DABCYL)-Norleucine-CONH2. The 4-dimethylaminoazobenzene-4′-carboxylic acid (DABCYL) component served as the FITC quencher. Catalytic domain of BoNT/A cleaves the peptide cleavage site is between its Gin and Arg residues, as highlighted in bold. The FRET substrate peptide was synthesized by New England Peptide (Gardener, Mass.) and possessed a purity >than 95%.

The peptide substrate stock solution was prepared using distilled water to obtain a 10 μM solution. BoNT/A LC is diluted with assay buffer to prepare a 100 nM stock solution. To carry out the screening assay, 50 μL of BoNT/A LC is transferred into 96-well clear bottom micro titer plates (Corning, Corning, N.Y.) and compounds 1-10 (5 mg/ml in DMSO) is transferred into each well. The final compound concentration is 25 μg/ml in each well. The natural compounds and BoNT/A LC were pre-incubated at 37° C. for 30 min. 50 μL peptide substrate is added to the reaction mixture of enzyme and inhibitor. Each plate contained at least 3 wells for positive controls and 3 wells for negative controls. The plates were incubated at 37° C. for 30 minutes to allow the endopeptidase reaction to occur. The positive control was BoNT/A LC without inhibitor but with the DMSO. The negative control was assay buffer without either BoNT/A LC or inhibitor. The plates were read using a SpectraMax M5 fluorescence microplate reader (Molecular Devices). The excitation wavelength used was 490 nm with an emission wavelength of 523 nm with auto cutoff.

The IC₅₀ value for small molecule inhibitors using FRET peptide substrate based endopeptidase assay and dose dependent inhibition curve was established. Using 50 nM LC of BoNT/A and different concentrations of the inhibitor incubated at 37° C. for 30 min before addition of the 5 μM of >95% purified substrate peptide. Enzyme and substrate mixture was incubated at 37° C. for 3 to 4 hours while reading plate every 30 min using excitation wavelength of 490 nm and emission wavelength of 523 nm. The IC₅₀ value was interpolated from the concentration response curve using a non-linear polynomial regression.

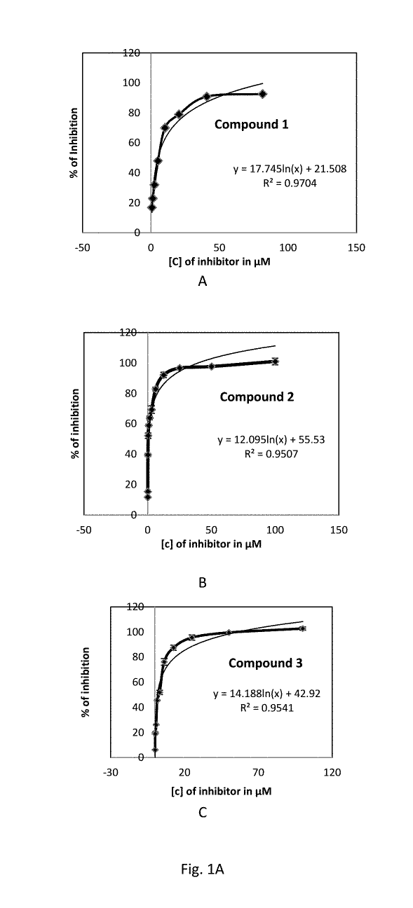

The small molecule inhibitors exhibited a dose-dependent inhibition effect against the endopeptidase activity of rLCA. The IC₅₀ value of small molecular inhibitor is in the low micro molar range. 10 different concentrations of inhibitor were used. Plots of [concentration of inhibitor] vs. % inhibition (FIG. 1) shown clear saturation points for nitro derivative of PNB (Compound 1) reached near 100% inhibition about 81 μM. Since the enzyme concentration we used in the inhibition assay is 50 nM, which means that 50% LCA inhibition is achieved when the molar ratio (inhibitor: rLCA) is 120:1 for the PNB. The IC₅₀ plots of all other 9 compounds also shown in FIG. 1. The determined IC₅₀ values using the peptide as a substrate has mentioned in the table 1.

Further, a 25 kDa full length recombinant SNAP-25 is attached with N-terminal-GST tag-SNAP25-GFP-C terminus (SNAG) fusion protein is prepared. GST is 26 kDa proteins and GFP is 27 kDa proteins. The 77 kDa fusion protein was purified.

Traditionally used method had limitation of the 1 kDa difference of uncleaved recombinant 25 kDa SNAP-25 proteins, with the cleavage product of 24-kDa protein. BoNT A and E cleavage sites are very close to C-terminus of SNAP-25 protein. The 77 kDa substrate protein construct is cleaved by LC of BoNT/A into 28 kDa and 49 kDa protein with the molecular weight difference of 21 kDa, which is very easily discerned on a SDS-PAGE gel. The N-terminal-GSTtag-SNAP25-GFP-C terminus (SNAG) was constructed in our lab. The endogenous GST of E. coli is about 22.9 kDa and the GFP tag used by SNAG is about 26.3 kDa. The intact SNAG is 77 kDa. Upon incubation SNAG is cleaved into 48.7 kDa and 28.3 kDa when incubated with LCA. The inhibitor is pre-incubated for 30 min at 37° C. with recombinant light chain A. After addition of SNAG the reaction mixture was incubated additionally for 30 min. The reaction was stopped by adding SDS running buffer to the reaction mixture followed by boiling the sample for 3 min in the water bath. The reaction results were examined and viewed by running the precast mini SDS-PAGE (420% TrisHCl, 10 wells, Bio-Rad Laboratories).

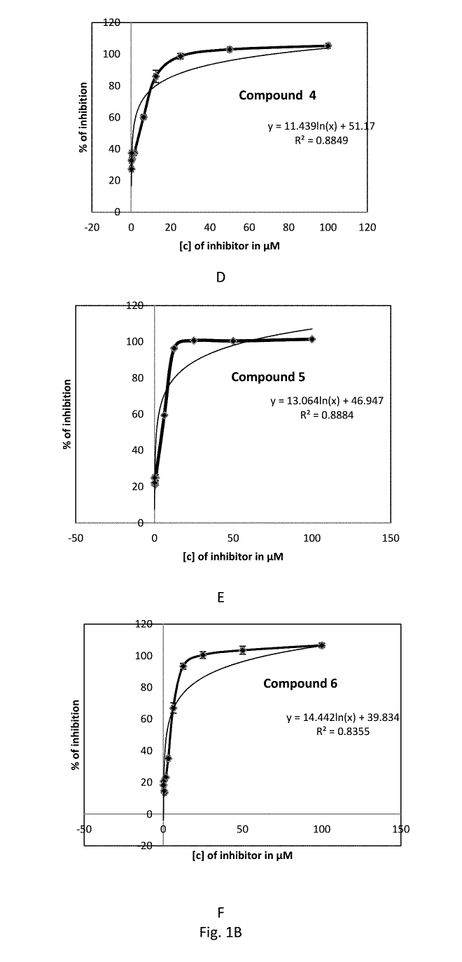

The inhibitors did present different level of inhibitions (FIG. 2). Low concentration of enzyme (rLCA) was used to slow down the cleavage rate to clearly observe inhibition activity during the reaction. The enzyme concentration that was used was as low as 2 nM. Several different groups of molar ratios (inhibitor to enzyme) were used to compare and assess the degree of inhibition to see if there is a saturation point for inhibition. The Inhibition data are similar to the FRET-Nutide based inhibition. The inhibition was determined by using densitometry analysis using the Kodak Imaging System. The inhibitor inhibited more than 50% of the SNAG at 0.72 μg/ml (2.3 μM) concentration. The IC₅₀ values for the inhibitor for endopeptidase activity against SNAG substrate are listed in Table 1.

Further, N-terminus His-SNAP25-GFP-C terminus (His-SNAG) was constructed. The intact His-SNAG is 54 kDa. Upon incubation with LCA the His-SNAG is cleaved into 24 kDa and 30 kDa. The IC₅₀ values for the all the compounds for endopeptidase activity against His-SNAG substrate are listed in Table 1.

| | | % of | | |
|---|---|---|---|---|
| Sr. | | Inhibition | Peptide assay | SNAG Assay |
| No. | Compounds | at 100 μM | IC₅₀ in μM | IC₅₀ |
| 1 | Compound 1 | 100 | 6.5 | 10 μM |
| 2 | Compound 2 | 100.63 | 0.65 ± 0.032 | 1.62 μM ± 0.65 |
| 3 | Compound 3 | 92.69 | 2.97 ± 0.048 | 0.86 μM ± 0.21 |
| 4 | Compound 4 | 100 | 3.15 ± 0.043 | 11.4 μM ± 2.8 |
| 5 | Compound 5 | 100 | 4.5 ± 0.035 | 13.5 μM ± 1.4 |
| 6 | Compound 6 | 100 | 4.5 ± 0.0091 | 11.6 μM ± 2.7 |
| 7 | Compound 7 | 90.49 | 5.06 ± 0.055 | >20 μM |
| 8 | Compound 8 | 85.13 | 6.8 ± 0.035 | >20 μM |
| 9 | Compound 9 | 83.28 | 9.3 ± 1.59 | >20 μM |
| 10 | Compound 10 | 103.72 | 0.27 ± 0.0049 | 239 nM ± 0.026 |

The enzyme kinetics was carried out using the 13-mer peptide based substrate.

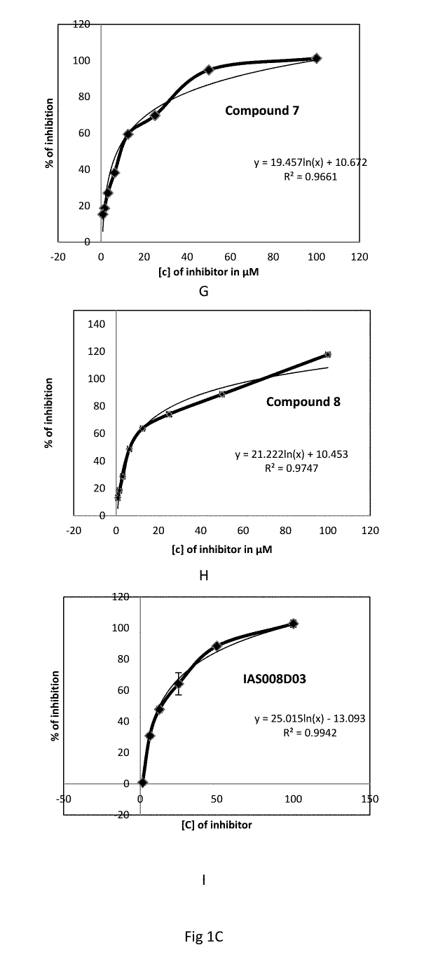

A series of concentrations from 5 to 25 μM of substrate were used (e.g., 5, 10, 20, and 25 μM) for enzyme kinetic study with 50 nM of LC of BoNT/A. The reaction buffer was same as the HTS assay described above. The reactions were carried out at 37° C., with the monitoring of fluorescence in the first 10 min to calculate the initial velocity of the reaction. The fluorescence was within the linear range for the contractions of substrate chosen above. To evaluate the inhibition kinetics, the inhibitor was pre-incubated with the LC of BoNT/A at 37° C. for 30 min before adding the substrate. The concentrations of inhibitor used were 19.5 and 39 μM. All the results are the average of triplicate measurements. The lineweaver-Burk plots were constructed for LCA endopeptidase activity against the peptide substrate, in the presence and absence of different concentrations of the inhibitor. The inhibitor showed (FIG. 3) a mixed (non competitive) type of inhibition, where, is KI<KI′ (Table 2). The non-competitive inhibition by PNB, showed an increase in Km values (20.5 μM, 24.0 μM, 30.5 μM, and 33.2 μM) and decrease in Vmax values (3.2, 2.8, 2.6, and 2.1 RFU/sec) with increase in the concentration (at 0, 6.25 μM, 12.5 μM, and 25 μM, respectively) of the inhibitor (Table 2). The non-competitive inhibition indicates that the inhibitor's enzyme binding site may be different from the substrate: enzyme binding site and it can interfere both enzyme active site and enzyme-substrate complex.

TABLE 2
Kinetic and inhibition constants (Vₘₐₓ (RFU/s), K_{M} (μM), α′, K_{I}′, α and K_{I}) for inhibitor PNB

| | Vmax (RFU/s) | | KM (μM) | | α′ | Ki′ value | α | Ki value |
|---|---|---|---|---|---|---|---|---|
| control | 3.17 | (±0.11) | 20.46 | (±1.88) | | | | |
| 6.25 μM | 2.79 | (±0.5) | 23.98 | (±5.1) | 1.155 (±0.18) | 40.32 (±1.0) | 1.38 (±0.33) | 17.48 (±3.1) |
| inhibitor | | | | | | | | |
| 12.5 μM | 2.55 | (±0.24) | 30.5 | (±4.9) | 1.24 (±0.12) | 52.08 (±2.5) | 1.8 (±0.30) | 14.7 (±6.8) |
| inhibitor | | | | | | | | |
| 25 μM | 2.064 | (±1.15) | 33.22 | ((±1.4) | 1.705 (±0.79) | 35.71 (±1.4) | 2.76 (±1.12) | 14.2 (±1.4) |
| Inhibitor | | | | | | | | |

The cytotoxicity of inhibitors was performed using MTT (3-(4,5-dimethylthizol-2-yl)-2,5-diphenyltetrazolium bromide) cell viability assay. SHSY-5Y cells or M17 cells were grown and maintained in a 5% CO2 and 95% humidity chamber at 37° C. in Dulbecco's Modified Eagle Medium (DMEM) containing 10% fetal bovine serum (FBS), kanamycin (100 μg/ml), penicillin (100 μg/ml) and streptomycin (100 μg/ml) antibiotics (Life Technologies, Grand Island, N.Y.). The 96 well L-lysine coated plates were used for the assay. Upon reaching 80% confluency, the media was washed and replenished with fresh serum free DMEM media without antibiotics. Cells were then incubated with different concentrations of inhibitors for 24 hours. For the positive control, six wells were used without inhibitors. Six empty wells without any cells were also used for the negative control to remove the background signal. Cell viability was measured after 24 hours using the MTT assay (ATCC, Manassas, Va.) in triplicates as recommended by the manufacturer. Absorbance was recorded at 570 nm with Molecular Devices SpectraMax Plus microplate reader. The relative viability of cells was normalized using the absorbance from positive and negative controls.

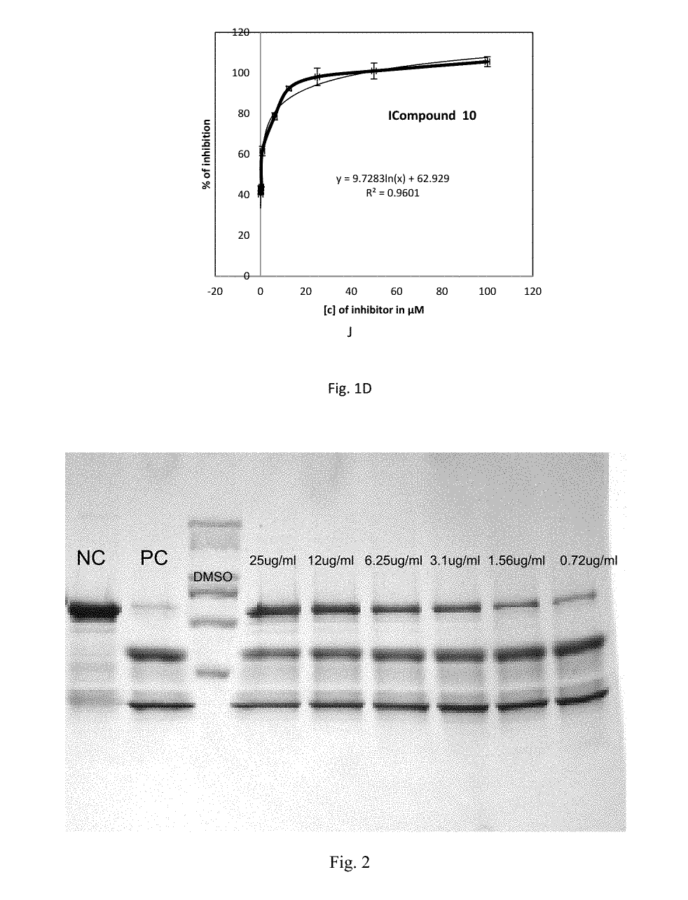

The cell viability was normalized using the untreated cells (positive control). The inhibitor when incubated for 24 hours reduced the cell viability of cultured M17 cells in a dose-dependent manner (FIG. 4). However, high concentration up to 310 μM of the inhibitor was well tolerated by the cells, retaining more than 90% cell viability. The M17 cells were incubated in DMEM media with 10% FBS. Cells were seeded in 12 well plates with 2.5×105 cells per well and maintained in a humidified 5% CO2 atmosphere at 37° C. Media was changed the next day and the cells were allowed to grow till 90% confluence. 30 nM BoNT/A and different Inhibitor concentrations were pre-incubated at 37° C. for 30 min. The positive control was kept with and without addition of the BoNT/A. The pre-incubated inhibitor and BoNT/A mixture were incubated for 36 hours with the cells. After 36 h incubation, the cells were washed with phosphate buffered saline (PBS) and harvested into a pre-weighed Eppendorf screw cap vial. The cells were then pelleted by centrifugation. After pelleting, the supernatant was carefully removed and the microcentrifuge tubes were incubated with 10 μl of mammalian protein extraction reagent (M-PER), and 10 μl of sodium dodecylsulfate-polyacrylamide gel electrophoresis (SDS-PAGE) sample buffer, and boiled for 10 minutes to inactivate the residual toxin. The samples were subsequently subjected to immunoblot analysis.

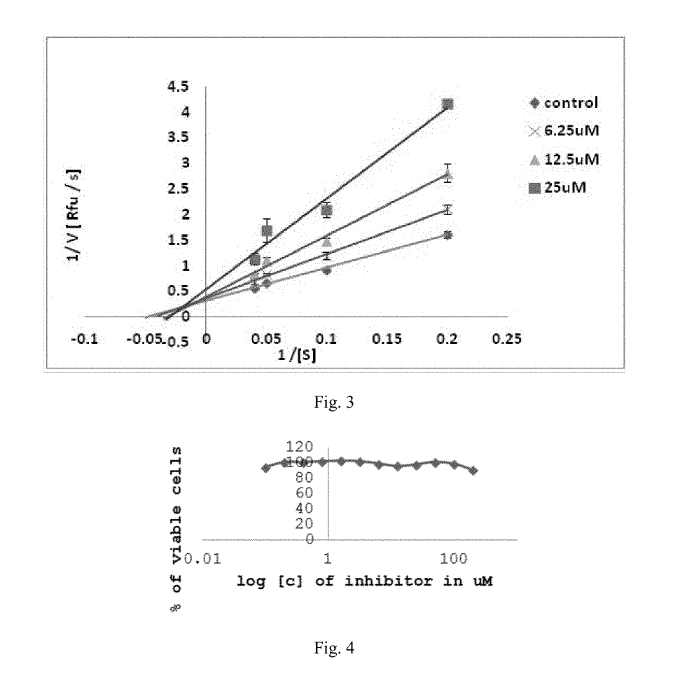

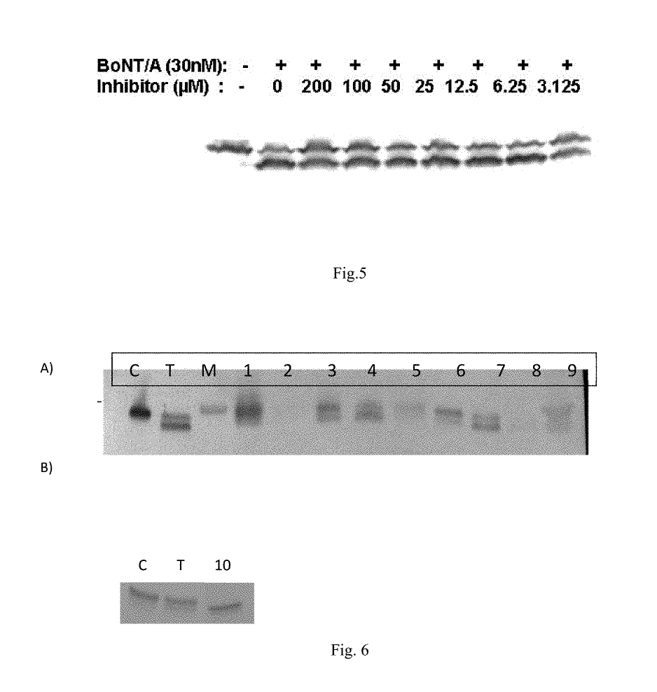

The aliquots of protein were electrophoresed through 15% polyacrylamide gels using Bio-rad precision plus protein Kaleidoscope standard (161-0375) the transfer of the bands to the PVDF membrane was examined. Proteins were transferred to polyvinylidene fluoride PVDF membranes (Millipore Corp., Bedford, Mass.) and processed for immunoblot analysis. The membrane was first blocked with 5% nonfat dry milk in TBST (10 mM Tris, 150 mM NaCl, 0.1% (v/v) Tween 20) for 1 h at room temperature, and the blots were probed with rabbit anti-SNAP-25 antibody (1:2500, Sigma, St. Louis, Mo.) in 1% nonfat dry milk in TBST for 1 h at room temperature. Following washes with TBST, the membrane was incubated with the secondary antibody, alkaline phosphatase conjugated anti-Rabbit IgG antibody (sigma) in 1% nonfat dry milk in TBST. After 1 h at room temperature, blots were washed and developed using BCIP (5-Bromo-4-Chloro-Indolyl-Phosphatase) stain. Reaction was stopped using distilled water washing of the membrane. Band densities for SNAP-25 and its cleaved product were normalized and relative intensities were determined using scanning densitometry (odyssey imager). The inhibition of the endopeptidase activity was examined with increasing concentrations of small molecule inhibitors. Since SHSY-5Y and M17 cell lines are less sensitive than primary cell cultures the experiments were carried out with higher concentrations of BoNT/A. To inhibit the higher concentration of the BoNT/A higher concentration of the inhibitor is required which can become toxic to the cells. Small molecular compound PNB showed inhibition at 200 μM and 100 μM (FIG. 5). Moreover compound 10, compound 9, compound 6, compound 3 and compound 1 (PNB) significantly inhibit endogenous SNAP-25 cleavage in M-17-TR2 cells (FIG. 6). According to FIG. 6 Western blot of SNAP-25 cleavage in M17 neuroblastoma cells. Lane 1, 2, 3, 4, 5, 6, 7, and 8 are extracted SNAP-25 from cell co-incubated with toxin and inhibitor. Lane C is control cells without toxin as a negative control and lane T is cells with 30 nM toxin as a positive control. Lane M indicated marker band at 25 kD. Lane 1 is from cells treated with compound 10+toxin, lane 2 is from cells treated with compound 4+toxin, lane 3 is from cells treated with compound 6+toxin, lane 4 is from cells treated with compound 2+toxin, lane 5 is from cells treated with compound 5+toxin, lane 6 is from cells treated with compound 3+toxin, lane 7 is from cells treated with compound 7+toxin, lane 8 is cells treated with compound 8+toxin, and lane 9 is cells treated with compound 9+toxin (panel A). Panel B is the blot of compound 1, PNB. Toxin and inhibitor concentration was 30 nM and 100 μM. C, T and M are negative control, positive control and marker, respectively. Negative control was cells without any treatment and positive control was cells incubated with 30 nM toxin.

### ADVANTAGE OF PRESENT INVENTION

Present invention discloses compounds that inhibit the toxic activity of BoTN.
The compounds are naturally occurring or synthetic derivatives or its pharmaceutically acceptable slats.

1. A method of treating an individual suffering from botulism comprising:
administering to the subject a composition comprising a therapeutically effective amount of compounds 1-10 or its pharmaceutically acceptable salts or derivatives either alone or combination thereof;
wherein the compound 1-10 are;

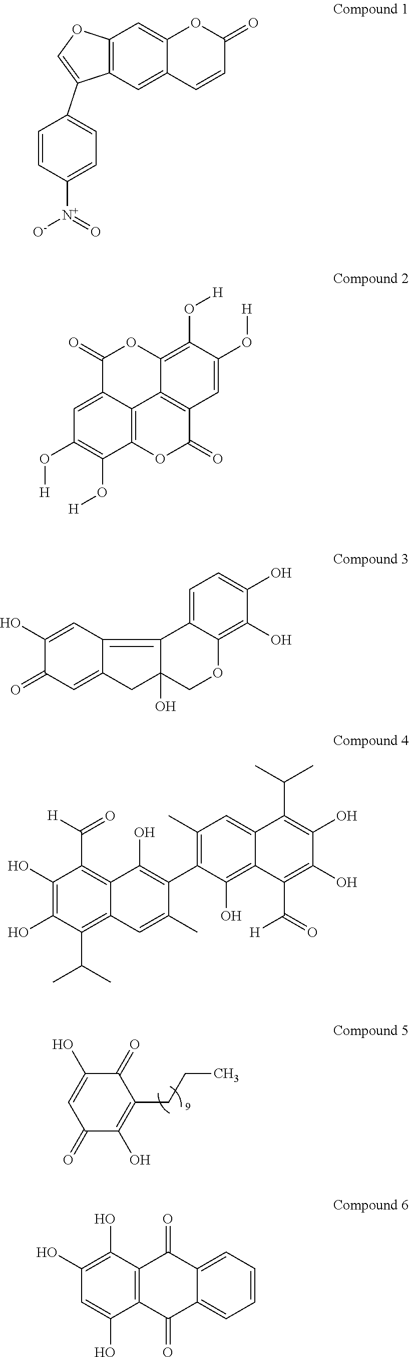

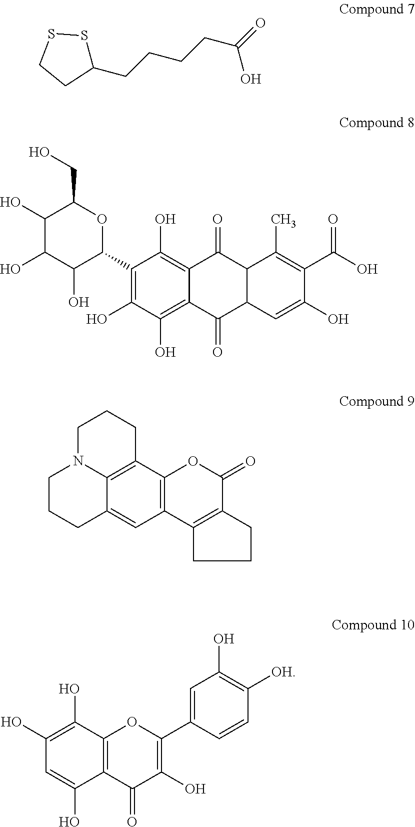 [SMILES: O=C(O)CCCCC1CCSS1 ; CC1=C(C(=O)O)C(O)=CC2C(=O)c3c(O)c(O)c([C@H]4O[C@H](CO)C(O)C(O)C4O)c(O)c3C(=O)C12 ; O=c1oc2c3c4c(cc2c2c1CCC2)CCCN4CCC3 ; O=c1c(O)c(-c2ccc(O)c(O)c2)oc2c(O)c(O)cc(O)c12]

2. The method of claim 1, wherein compounds 1-10 optionally with pharmaceutically acceptable diluent, excipient or carrier

3. The method of claim 1, wherein the composition further comprises pharmaceutically acceptable binder(s), lubricant(s), suspending agent(s), coating agent(s), solubilizing agent(s).

4. The method of claim 1 wherein the compound 1-10 further delivered in combination with liposomes or nanoparticle linked to detoxified recombinant BoNT.

5. The method of claim 1, wherein the botulism caused by botulinum neurotoxin produced by clostridium botulinum.

6. The method of claim 1, wherein the compounds 1-10 inhibit botulinum neurotoxin.

7. The method of claim 4, wherein the botulinum neurotoxin is type A, B, C, D, E, F, G.

8. The method of claim 4, wherein the botulinum neurotoxin is more preferably A, B, E and F.

9. The method of claim 2, wherein the diluent is selected from the group consisting of ethanol, glycerol, DMSO water or mixture thereof.

10. The method of claim 2, wherein the carrier is selected from group consisting of lactose, starch, glucose, methyl cellulose, magnesium stearate, mannitol, sorbitol.

11. The method of claim 3, wherein the binders selected from the group consisting of starch, gelatin, natural sugars such as glucose, anhydrous lactose, free-flow lactose, beta-lactose, corn sweeteners, natural and synthetic gums.

12. The method of claim 11, wherein the natural and synthetic gums selected from the group consisting of acacia, tragacanth or sodium alginate, carboxymethyl cellulose and polyethylene glycol.

13. The method of claim 3, wherein the lubricant is selected from the group consisting of sodium oleate, sodium stearate, magnesium stearate, sodium benzoate, sodium acetate, sodium chloride.

14. The method of claim 1, wherein the therapeutically effective amount is in the range of <1 to 10 μM/Kg.

15. The method of claim 1, wherein the composition is administered by oral administration, nasal administration, topical administration, parenteral administration, rectal administration, systemic administration, intramuscular administration, or intravenous administration.

16. The method of claim 1, wherein the composition is administered more preferably by oral administration or nasal administration.
